(19) United States
(12) Patent Application Publication
(10) Pub. No.: US 2025/0009732 A1
(43) Pub. Date: Jan. 9, 2025

(54) DISSOLVABLE ORALLY ADHERING NICOTINE TABLET

(71) Applicant: Fertin Pharma A/S, Vejle (DK)

(72) Inventors: Helle Wittorff, Vejle (DK); Bruno Provstgaard Nielsen, Vejle (DK); Helle Martinussen, Vejle (DK)

(21) Appl. No.: 18/766,023

(22) Filed: Jul. 8, 2024

(30) Foreign Application Priority Data
Jul. 7, 2023 (DK) PA 2023 70372
Jul. 7, 2023 (DK) PA 2023 70373
Nov. 7, 2023 (DK) PA 2023 30319

(51) Int. Cl. A61K 31/465; A61K 9/00; A61K 9/20; A61K 9/24

(52) U.S. Cl. CPC A61K 31/465; A61K 9/0056; A61K 9/2009; A61K 9/2018; A61K 9/205; A61K 9/2054; A61K 9/2086; A61K 9/209

(57) ABSTRACT

A dissolvable orally adhering nicotine tablet for sustained delivery of nicotine is disclosed, the tablet is a multilayer tablet comprising a first layer and a second layer, wherein the first layer is a mucoadhesive layer comprising a mucoadhesive, nicotine, and at least one sugar alcohol, and wherein the second layer comprises at least one sugar alcohol.

### FIELD OF INVENTION

The invention relates to a dissolvable orally adhering nicotine tablet for sustained release according to the claims.

### BACKGROUND

Several oral tablets are known to provide sustained release of nicotine. However, such tablets may be inconvenient for the user due to the slow dissolution of the tablets. Additionally, slow dissolution may result in an unintended swallowing of nicotine, which further would result in an unpleasant experience for the user. Besides being an unpleasant experience, unintended swallowing would prohibit buccal release of nicotine. Thus, a lowered efficacy and undesired side effects such as off-taste, throat irritation and/or hiccups may be expected.

At the same time, due to the slow dissolution, providing tablets with a good mouthfeel is desirable, as users may otherwise discourage from use, or may eject the tablets prematurely due to discomfort.

It is an object of the present invention to overcome the disadvantages of the prior art.

### SUMMARY

The invention relates to a dissolvable orally adhering nicotine tablet for sustained delivery of nicotine, wherein the tablet is a multilayer tablet comprising a first layer and a second layer,
wherein the first layer is a mucoadhesive layer comprising a mucoadhesive, nicotine, and at least one sugar alcohol, and
wherein the second layer comprises at least one sugar alcohol.

An advantage of the invention may be that a desirable sustained delivery of nicotine may be obtained.

An advantage of the present invention may be that a localized release of nicotine may be obtained. Due to the mucoadhesive layer, the tablet may be immobilized in the intended position between the gum and the lip, while the second layer facilitates adherence towards one side only, thus preventing unpleasant adherence between the gum and lip. It is noted that the tablet preferably is inserted to adhere to the mucosal surface of the gum.

Furthermore, due to the effective adherence facilitated by the mucoadhesive layer, a convenient, pleasurable user experience is provided. First of all, a more discreet nicotine delivery format is provided compared to other common nicotine delivery formats such as chewing gum, lozenges and pouch. Not only since unintended appearance of the tablet from its gum-lip position may be prevented, but also since the tablet size may be reduced. Typically, smaller tablets positioned between the gum and the lip may easily dislocate from their intended position and may thus be unintentionally chewed or swallowed or even fall out of the mouth. Such unintended dislocation may also result in excessive nicotine and buffer irritation in the mouth and throat due to faster than intended release of nicotine and/or buffer followed by swallowing. In turn, this also leads to inefficient utilization of nicotine. Adhering tablets may, however, also be perceived as unpleasant, as the user may often perceive the tablet as holding the gum and the lip together due to the adhesive nature of the tablet.

Furthermore, an advantage of the invention may be that the release of nicotine from the mucoadhesive layer is facilitated by the presence of the at least one sugar alcohol in the mucoadhesive layer, while the sugar alcohol also decreases dissolution time to a desired time period for sustained release.

The present invention provides a discreet tablet, which may be smaller than typical tablets for intraoral use and thus avoid any unpleasant bulky sensation. At the same time, the tablet may be firmly adhered to the gum or lip to avoid unintentional dislocation of the tablet, yet avoid the disadvantages typically associated with adherent tablets, due to the multilayer design comprising a second layer with sugar alcohol and less adhesive properties. These advantages may be obtained without requiring any subsequent user action, such as special use, removal of residual product etc.

A further advantage of the invention may be that a sustained release of nicotine may be obtained in a simple manner.

Specifically, by including a mucoadhesive selectively in the first layer, the tablet may after insertion between the gum and the lip adhere to the mucosal surface of the gum or to the lip, preferably to the gum, whereby the tablet is immobilized. This immobilization facilitates sustained release of nicotine, as the tablet is inserted into a low exposure region of the oral cavity with low saliva flux and minimum interaction with the tongue. Thereby the disintegration time of the tablet may be significantly prolonged. On the other hand, the adhesion either to the mucosal surface of the gum or to the lip facilitates a significant improvement in the user experience, as simultaneous adhesion to the mucosal surface of the gum and the lip is perceived as unpleasant, e.g. since it prevents the gum and the lip from moving freely relative to each other. Based on this insight, the tablet comprises both a first layer being a mucoadhesive layer and a second layer comprising sugar alcohol.

It is noted that the intended use of the tablet is insertion between the gum and the lip, with the first layer facing the gum or the lip, preferably the gum, whereby the first layer adheres to the gum or lip to facilitate immobilization of the tablet. Thus, the layered structure of the tablet with the mucoadhesive layer on one side facilitates this advantageous use.

It is noted within the present context that the term “adhering tablet” refers to a tablet which is able to adhere to a mucosal surface of the oral cavity, particularly the gum or lip, preferably the gum, by means of the mucoadhesive layer.

It is noted within the present context that the term “sustained release” refers to release over a prolonged period of time, in particular over period of at least 15 minutes, such as at least 30 minutes, such as at least 1 hour. It is noted that sustained release may sometimes also be referred to as controlled release or extended release. According to an embodiment of the invention, the tablet is adapted for release of the nicotine over a prolonged period of time, such as 15 minutes to 6 hours, such as 30 minutes to 2 hours, or such as 1 to 10 hours, such as 2 to 8 hours, such as 3 to 6 hours. The sustained release, i.e. the sustained release of the nicotine, may be obtained by the tablet being an orally adhering tablet, i.e. suitable for providing sustained release of the nicotine over a prolonged period of time by the tablet adhering to mucosal surface of the oral cavity, particularly the gum or lip. In an embodiment of the invention, the sustained release should not be understood as limiting, and in such embodiment the invention relates to a dissolvable orally adhering nicotine tablet for sustained delivery of nicotine, wherein the tablet is a multilayer tablet comprising a first layer and a second layer, wherein the first layer is a mucoadhesive layer comprising a mucoadhesive, nicotine, and at least one sugar alcohol, and wherein the second layer comprises at least one sugar alcohol.

It is noted within the present context that the term “mucoadhesive layer” refers to the first layer comprising mucoadhesive, whereby the first layer may adhere to a mucosal surface of the oral cavity, particularly the gum or lip, preferably the gum. It is noted that due to the tablet being inserted between the gum and the lip, the mucoadhesive effect should facilitate adhesion despite the squeezing or clamping effect from the gum and the lip, preferably such that the tablet is immobilized during normal behavior of the user, including e.g. eating and drinking. Thus, the term “mucoadhesive layer” and “first layer” may be used interchangeably. It is further noted that the first layer is at least partly exposed such that it is able to come into contact with an oral mucosal surface, such as the gum or lip, in order to facilitate adhesion thereto. In an embodiment of the invention, a compound having mucoadhesive properties is considered to be a mucoadhesive when present in an amount of at least 15%. Thus, if being present in lower amounts, e.g. 5-10%, it may be considered a binder or dissolution modifier.

It is noted within the present context that the term “second layer” refer to a layer that does not provide the adhesive effect of the first layer. Thus, the second layer may act as a non-adhesive layer to allow the tablet to specifically adhere to only one side, whereby the gum and lip may move freely relative to each other.

According to an advantageous embodiment of the invention, the tablet is a compressed tablet.

In the present context, the term “compressed” refers to formed by compression from a plurality of particles and/or granules, such as by means of a tableting machine, such as e.g. a rotary press. The tablet of the above embodiment is a compressed tablet formed by compression of at least a first powdered composition and a second powdered composition to give the first and second layers, respectively. Typically, the tablet may be made in a multi-step process, by compressing the first or second layer first, then sequentially compressing the other layers to obtain a multilayer tablet. When the tablet is a two-layer tablet, it may be made in a two-step process, by compressing the first or second layer, then compressing the other layer to obtain the two-layer tablet.

In an embodiment of the invention, the first layer is compressed.

In an embodiment of the invention, the second layer is compressed.

According to an advantageous embodiment of the invention, the tablet is composed of a plurality of compressed particles.

In an embodiment of the invention, the first layer is composed of a plurality of compressed particles.

In an embodiment of the invention, the second layer is composed of a plurality of compressed particles.

According to an advantageous embodiment of the invention, the first and the second layers are fused by compression.

According to an advantageous embodiment of the invention, the mucoadhesive comprises a mucoadhesive selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, hydroxypropyl cellulose (HPC), hydroxypropyl methylcellulose (HPMC), hydroxy ethylcellulose, ethylcellulose (EC), carboxymethyl cellulose (CMC) and salts thereof, dextran, guar gum, polyvinyl pyrrolidone (PVP), gelatin, casein, acrylic acid polymers (carbomers), acrylic acid esters, acrylic acid copolymers, and any combination thereof.

According to an embodiment of the invention, the mucoadhesive comprises a mucoadhesive selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, retene, agarose, hydroxypropyl cellulose (HPC), hydroxypropyl methylcellulose (HPMC), hydroxy ethylcellulose, ethylcellulose (EC), carboxymethyl cellulose (CMC) and salts thereof, dextran, guar gum, polyvinyl pyrrolidone (PVP), gelatin, casein, acrylic acid polymers (carbomers), acrylic acid esters, acrylic acid copolymers, poly vinyl alcohol, poly hydroxyethyl methylacrylate, poly ethylene oxide, hydroxy ethyl cellulose, hydroxy propyl cellulose, methyl cellulose, chitosan, soluble starch, lecithin, and any combination thereof.

According to an advantageous embodiment of the invention, the mucoadhesive comprises an ionic mucoadhesive.

As used herein, the term “ionic mucoadhesive” refers to ionizable polysaccharide mucoadhesives, sometimes also referred to as polyelectrolytes. Thus, ionic mucoadhesive does not include e.g. polyvinyl pyrrolidone.

An advantage of the above embodiment may be that the release profile of the active ingredient may be altered/modified.

In an embodiment of the invention, the mucoadhesive consists of an ionic mucoadhesive.

According to an advantageous embodiment of the invention, the mucoadhesive comprises a mucoadhesive selected from the group consisting of xanthan gum, gellan gum, gum arabic, alginic acid, alginate, carrageenan, agar, and any combination thereof.

In an embodiment of the invention, the mucoadhesive is selected from the group consisting of xanthan gum, konjac gum, gellan gum, gum arabic, alginate, carrageenan, agar, and any combination thereof.

According to an advantageous embodiment of the invention, the mucoadhesive comprises xanthan gum and/or gum arabic.

According to an advantageous embodiment of the invention, the mucoadhesive comprises gum arabic.

In an embodiment of the invention, the mucoadhesive consists of gum arabic.

According to an advantageous embodiment of the invention, the mucoadhesive comprises xanthan gum.

In an embodiment of the invention, the mucoadhesive consists of xanthan gum.

In an embodiment of the invention, the mucoadhesive comprises a natural gum.

In an embodiment of the invention, the mucoadhesive consists of a natural gum.

In an embodiment of the invention, the mucoadhesive comprises a natural gum mucoadhesive selected from the group consisting of xanthan gum, konjac gum, tara gum, gellan gum, locust bean gum, gum arabic, alginate, pullulan, tragacanth gum, gum karaya, fenugreek gum, cassia gum, carrageenan, agar, alginic acid and salts thereof, and any combination thereof.

In an embodiment of the invention the mucoadhesive is selected from the group consisting of xanthan gum, konjac gum, tara gum, gellan gum, locust bean gum, gum arabic, pullulan, tragacanth gum, gum karaya, fenugreek gum, cassia gum, and any combination thereof.

In an embodiment of the invention, the mucoadhesive comprises at least one natural gum selected from the group consisting of gum arabic, guar gum, locust bean gum, gellan gum, xanthan gum, and any combination thereof.

In an embodiment of the invention, the natural gum is selected from the group consisting of gum arabic, guar gum, locust bean gum, gellan gum, xanthan gum, and any combination thereof.

According to an advantageous embodiment of the invention, the mucoadhesive comprises a cellulose derivative.

An advantage of the above embodiment may be that the mucoadhesive provides a gel like structure at the end of dissolution of the tablet. To some users the gel like structure may be experienced as preferable compared to mucoadhesives not comprising cellulose derivatives, as the latter may provide a more rigid structure during dissolution of the tablet. Without being bound by theory it is contemplated that the gel like structure occurs as a result of excessive swelling of the cellulose derivative upon prolonged exposure to saliva.

According to an advantageous embodiment of the invention, the mucoadhesive comprises a cellulose derivative selected from the group consisting of hydroxypropyl cellulose (HPC), hydroxypropyl methylcellulose (HPMC), hydroxy ethylcellulose, ethylcellulose (EC), carboxymethyl cellulose (CMC), and salts thereof, and any combination thereof.

According to an advantageous embodiment of the invention, the first layer comprises a cellulose derivative mucoadhesive in an amount of at least 20% by weight of the first layer.

According to an advantageous embodiment of the invention, the first layer comprises at least two different types of mucoadhesives.

According to an advantageous embodiment of the invention, the mucoadhesive of the first layer comprises at least one natural gum and a least one cellulose derivative.

According to an advantageous embodiment of the invention, the mucoadhesive of the first layer comprises gum arabic and a least one cellulose derivative.

According to an advantageous embodiment of the invention, the first layer comprises the mucoadhesive in an amount of at least 40% by weight of the first layer, such as at least 50% by weight of the first layer, such as at least 55% by weight of the first layer, such as at least 60% by weight of the first layer.

According to an advantageous embodiment of the invention, the first layer comprises the mucoadhesive in an amount of at least 35% by weight of the first layer, such as at least 40% by weight of the first layer, such as at least 50% by weight of the first layer, such as at least 55% by weight of the first layer, such as at least 60% by weight of the first layer.

An advantage of the above embodiment may be that the tablet efficiently adheres to the gum.

According to an advantageous embodiment of the invention, the first layer comprises the mucoadhesive in an amount of no more than 90% by weight of the first layer, such as no more than 85% by weight of the first layer, such as no more than 80% by weight of the first layer.

An advantage of the above embodiment may be that it facilitates the eventual dissolution of the first layer. Having a sufficiently high content of mucoadhesive may be desirable to ensure adhesion of the table to the gum, however, a too high content of mucoadhesive may result in the first layer having an undesirable long dissolution time far exceeding the use time desired by the user.

In an embodiment of the invention, the first layer comprises the mucoadhesive in an amount of 35 to 90% by weight of the first layer, such as 40 to 90% by weight of the first layer, such as 50 to 90% by weight of the first layer, such as 55 to 85% by weight of the first layer, such as 60 to 80% by weight of the first layer.

According to an advantageous embodiment of the invention, the first layer comprises gum arabic in an amount of at least 35% by weight of the first layer, such as at least 40% by weight of the first layer, such as at least 50% by weight of the first layer, such as at least 55% by weight of the first layer, such as at least 60% by weight of the first layer.

In an embodiment of the invention, the first layer comprises gum arabic in an amount of 35 to 90% by weight of the first layer, such as 40 to 90% by weight of the first layer, such as 50 to 90% by weight of the first layer, such as 55 to 85% by weight of the first layer, such as 60 to 80% by weight of the first layer.

According to an embodiment of the invention, the second layer comprises less than 20% by weight of mucoadhesive, such as less than 15% by weight of mucoadhesive, such as less than 10% by weight of mucoadhesive, such as less than 5% by weight of mucoadhesive, such as less than 1% by weight of mucoadhesive, such as less than 0.2% by weight of mucoadhesive, such as being substantially free of mucoadhesive.

According to an embodiment of the invention, the second layer comprises 0% to less than 20% by weight of mucoadhesive, such as 0.01% to less than 10% by weight of mucoadhesive, such as 0.02% to less than 5% by weight of mucoadhesive, such as 0.05% to less than 1% by weight of mucoadhesive, such as 0.1% to less than 0.2% by weight of mucoadhesive, such as being substantially free of mucoadhesive.

In an embodiment of the invention, the second layer comprises mucoadhesive selected from xanthan gum, alginate and combinations thereof.

It is noted that if the second layer comprises certain mucoadhesive(s) such as xanthan gum and/or alginate in relatively low amounts as in the above embodiments, the mucoadhesive may also be regarded as a dissolution modifier slowing down dissolution of the second layer and/or release of nicotine from the second layer.

According to an advantageous embodiment of the invention, the second layer is free of mucoadhesive.

In an embodiment of the invention, the second layer is provided as a non-adhering layer due to its low content or absence of mucoadhesive.

In an embodiment of the invention, the first layer comprises less of less than 20% by weight of the first layer of substances selected from the group consisting of gelatin, chitosan, carboxymethyl cellulose and its sodium salt, hydroxypropylmethyl cellulose, polyvinyl alcohol, gellan gum, agar, agarose, and a combination thereof, such as less than 10% by weight of the first layer, such as less than 5% by weight of the first layer, such as less than 2% by weight of the first layer, such as less than 1% by weight of the first layer, such as 0% by weight of the first layer.

In an embodiment of the invention, the first layer is free of substances selected from the group consisting of gelatin, chitosan, carboxymethyl cellulose and its sodium salt, hydroxypropylmethyl cellulose, polyvinyl alcohol, gellan gum, agar, agarose, and a combination thereof.

In an embodiment of the invention, the first layer comprises less of less than 20% by weight of the first layer of substances selected from the group consisting of gelatin; pectin; starch; cellulose derivatives; agarose; chitosan and chitosan derivatives; hyaluronic acid; polyvinylpyrrolidone (PVP); polyacrylates; polyhydroxyethylmetacrlylate (PHEMA); polyvinylalcohol (PVA); lectins; thiolated polymers; polyethylene glycol (PEG); sodium alginate; guar gum; karya gum; gellan gum; agar; retene; tragacanth; and a combination thereof, such as less than 10% by weight of the first layer, such as less than 5% by weight of the first layer, such as less than 2% by weight of the first layer, such as less than 1% by weight of the first layer, such as 0% by weight of the first layer.

In an embodiment of the invention, the first layer is free of substances selected from the group consisting of gelatin; pectin; starch; cellulose derivatives; agarose; chitosan and chitosan derivatives; hyaluronic acid; polyvinylpyrrolidone (PVP); polyacrylates; polyhydroxyethylmetacrlylate (PHEMA); polyvinylalcohol (PVA); lectins; thiolated polymers; polyethylene glycol (PEG); sodium alginate; guar gum; karya gum; gellan gum; agar; retene; tragacanth; and a combination thereof.

In an embodiment of the invention, the first layer is free of hydroxypropyl methylcellulose.

In an embodiment of the invention, the first layer is free of carboxymethylcellulose.

In an embodiment of the invention, the first layer is free of cellulose derivates.

According to an advantageous embodiment of the invention, the tablet comprises nicotine in an amount of at least 0.2 mg, such as at least 0.5 mg, such as at least 1.0 mg.

In an embodiment of the invention, the tablet comprises nicotine in an amount of 0.2 mg to 12.0 mg, such as 0.2 mg to 10.0 mg, such as 0.5 mg to 8.0 mg, such as 0.5 mg to 6.0 mg, such as 1.0 mg to 4.0 mg, such as 1.0 to 3.0 mg.

According to an advantageous embodiment of the invention, the tablet comprises nicotine in an amount of at least 0.2% by weight of the tablet, such as at least 0.3% by weight of the tablet, such as at least 0.5% by weight of the tablet.

In an embodiment of the invention, the tablet comprises nicotine in an amount of 0.2 to 5% by weight of the tablet, such as 0.3 to 3% by weight of the tablet, such as 0.5 to 2% by weight of the tablet.

In an embodiment of the invention, the first layer comprises nicotine in an amount of at least 0.2 mg, such as at least 0.5 mg, such as at least 1.0 mg.

In an embodiment of the invention, the first layer comprises nicotine in an amount of 0.2 mg to 10.0 mg, such as 0.2 mg to 5.0 mg, such as 0.5 mg to 4.0 mg, such as 1.0 mg to 3.0 mg, such as 1.0 to 2.0 mg.

In an embodiment of the invention, the first layer comprises nicotine in an amount of at least 0.2% by weight of the first layer, such as at least 0.3% by weight of the first layer, such as at least 0.5% by weight of the first layer.

In an embodiment of the invention, the first layer comprises nicotine in an amount of 0.2 to 5% by weight of the first layer, such as 0.3 to 3% by weight of the first layer, such as 0.5 to 2% by weight of the first layer.

In an embodiment of the invention, the second layer comprises nicotine in an amount of at least 0.2 mg, such as at least 0.5 mg, such as at least 1.0 mg.

In an embodiment of the invention, the second layer comprises nicotine in an amount of 0.2 mg to 3.0 mg, such as 0.5 mg to 3.0 mg, such as 1.0 mg to 3.0 mg, such as 1.0 to 2.0 mg.

In an embodiment of the invention, the second layer comprises nicotine in an amount of at least 0.2% by weight of the second layer, such as at least 0.3% by weight of the second layer, such as at least 0.5% by weight of the second layer.

In an embodiment of the invention, the second layer comprises nicotine in an amount of 0.2 to 3% by weight of the second layer, such as 0.3 to 2.5% by weight of the second layer, such as 0.5 to 2% by weight of the second layer.

According to an advantageous embodiment of the invention, the nicotine is comprised in the first layer.

Thus, in the above embodiment, the first layer comprises all nicotine of the tablet, and the second layer is free of nicotine.

According to an advantageous embodiment of the invention, the second layer comprises nicotine.

Thus, in the above embodiment, the second layer comprises at least a part of the nicotine.

In an embodiment of the invention, the second layer is free of nicotine.

According to an advantageous embodiment of the invention, the nicotine is selected from the group consisting of a nicotine salt, nicotine free base, a nicotine-ion exchange resin combination, a nicotine inclusion complex or nicotine in any non-covalent binding; nicotine bound to zeolites, nicotine bound to cellulose, such as microcrystalline cellulose, starch microspheres, and mixtures thereof.

According to an advantageous embodiment of the invention, the nicotine comprises nicotine free base.

In an embodiment of the invention, the nicotine comprises nicotine free base.

According to an advantageous embodiment of the invention, the nicotine comprises nicotine bound to an ion exchange resin.

In an embodiment of the invention, the nicotine comprises nicotine bound to an ion exchange resin.

In an embodiment of the invention, the nicotine consists of nicotine bound to an ion exchange resin.

In an embodiment of the invention, the ion exchange resin comprises one or more resin(s) selected from the group consisting of:
(i) a methacrylic, weakly acidic type of resin containing carboxylic functional groups,
(ii) a copolymer of methacrylic acid and divinylbenzene, said copolymer containing carboxylic functional groups,
(iii) a polystyrene, strongly acidic type of resin containing sulphonic functional groups,
(iv) a polystyrene, intermediate acidic type of resin containing phosphonic functional groups, and
(v) a combination thereof.

According to an advantageous embodiment of the invention, the ion exchange resin comprises polacrilex resin.

According to an advantageous embodiment of the invention, the ion exchange resin is polacrilex resin.

The term NPR may be used as an abbreviation to denote nicotine complexed with the ion-exchange resin specifically known as polacrilex resin.

According to an advantageous embodiment of the invention, the nicotine comprises a nicotine salt.

In an embodiment of the invention, the nicotine consists of one or more nicotine salts.

In an embodiment of the invention, the nicotine salt is selected from nicotine ascorbate, nicotine aspartate, nicotine benzoate, nicotine monotartrate, nicotine bitartrate, nicotine chloride (e.g., nicotine hydrochloride and nicotine dihydrochloride), nicotine citrate, nicotine fumarate, nicotine lactate, nicotine mucate, nicotine laurate, nicotine levulinate, nicotine malate nicotine perchlorate, nicotine pyruvate, nicotine salicylate, nicotine sorbate, nicotine succinate, nicotine sulfate, hydrates thereof, or any combination thereof.

According to an advantageous embodiment of the invention, the nicotine salt comprises nicotine bitartrate.

The term NBT may be used as an abbreviation to denote nicotine bitartrate.

According to an embodiment of the invention, the nicotine salt consists of nicotine bitartrate.

In an embodiment of the invention, the nicotine comprises synthetic nicotine.

In an embodiment of the invention, the nicotine consists of synthetic nicotine.

According to an advantageous embodiment of the invention, the tablet has a dissolution time of at least 15 minutes, such as at least 20 minutes, such as at least 30 minutes, such as at least 45 minutes.

According to an advantageous embodiment of the invention, the tablet has a dissolution time of no more than 2.5 hours, such as no more than 2 hours, such as no more than 1.5 hours, such as no more than 1 hour.

In an embodiment of the invention, the tablet has a dissolution time of no more than 10 hours, such as no more than 8 hours, such as no more than 5 hours, such as no more than 4 hours.

In an embodiment of the invention, the tablet has a dissolution time of 15 minutes to 2.5 hours, such as 20 minutes to 2 hours, such as 30 minutes to 2 hours, such as 45 minutes to 1.5 hour, such as 45 minutes to 1 hour.

In an embodiment of the invention, the first layer has a dissolution time of at least 15 minutes, such as at least 20 minutes, such as at least 30 minutes, such as at least 45 minutes.

In an embodiment of the invention, the first layer has a dissolution time of no more than 2.5 hours, such as no more than 2 hours, such as no more than 1.5 hours, such as no more than 1 hour.

In an embodiment of the invention, the first layer has a dissolution time of 15 minutes to 2.5 hours, such as 20 minutes to 2 hours, such as 30 minutes to 2 hours, such as 45 minutes to 1.5 hour, such as 45 minutes to 1 hour.

In an embodiment of the invention, the second layer has a dissolution time of at least 15 minutes, such as at least 20 minutes, such as at least 30 minutes, such as at least 45 minutes.

In an embodiment of the invention, the second layer has a dissolution time of no more than 2.5 hours, such as no more than 2 hours, such as no more than 1.5 hours, such as no more than 1 hour.

In an embodiment of the invention, the second layer has a dissolution time of 15 minutes to 2.5 hours, such as 20 minutes to 2 hours, such as 30 minutes to 2 hours, such as 45 minutes to 1.5 hour, such as 45 minutes to 1 hour.

In an embodiment of the invention, the dissolution time of the above embodiments is in vitro dissolution time.

In vitro dissolution time measurements are carried out in accordance to European Pharmacopeia 9.0, section 2.9.1, Disintegration of tablets and capsules.

In an embodiment of the invention, the dissolution time of the above embodiments is in vivo dissolution time.

According to an embodiment of the invention, the in vivo dissolution time was measured by at least 6 trained assessors, the trained assessors abstaining from eating and drinking at least 30 minutes before initiation of any test, the tablet was weighted and placed in the mouth, between the upper lip and the gum with the first facing the gum, where the in vivo dissolution time was registered as the time point where the tablet dissolution was substantially complete.

According to an embodiment of the invention, in vivo dissolution time was measured in accordance with example 4.

According to an advantageous embodiment of the invention, the tablet comprises a pH regulating agent, such as an alkaline pH regulating agent, such as an alkaline buffering agent.

In an embodiment of the invention, the first layer comprises a pH regulating agent.

In an embodiment of the invention, the pH regulating agent of the first layer is an alkaline pH regulating agent, such as an alkaline buffering agent.

In an embodiment of the invention, the second layer comprises a pH regulating agent.

In an embodiment of the invention, the pH regulating agent of the second layer is an alkaline pH regulating agent, such as an alkaline buffering agent.

In an embodiment of the invention, the pH regulating agent is a buffering agent, such as an alkaline buffering agent.

According to an advantageous embodiment of the invention, the tablet comprises the pH regulating agent in an amount of at least 0.2% by weight of the tablet, such as at least 0.5% by weight of the tablet, such as at least 1% by weight of the tablet, such as at least 2% by weight of the tablet.

In an embodiment of the invention, the tablet comprises the pH regulating agent in an amount of 0.2 to 10% by weight of the tablet, such as 0.5 to 8% by weight of the tablet, such as 1 to 6% by weight of the tablet, such as 2 to 4% by weight of the tablet.

According to an advantageous embodiment of the invention, the pH regulating agent is comprised in the first layer.

In an embodiment of the invention, the first layer comprises the pH regulating agent in an amount of at least 0.2% by weight of the first layer, such as at least 0.5% by weight of the first layer, such as at least 1% by weight of the first layer, such as at least 2% by weight of the first layer.

In an embodiment of the invention, the first layer comprises the pH regulating agent in an amount of 0.2 to 10% by weight of the first layer, such as 0.5 to 8% by weight of the first layer, such as 1 to 6% by weight of the first layer, such as 2 to 4% by weight of the first layer.

In an embodiment of the invention, the second layer is free of pH regulating agent.

According to an advantageous embodiment of the invention, the pH regulating agent is comprised in the second layer.

In an embodiment of the invention, the second layer comprises the pH regulating agent in an amount of at least 0.2% by weight of the second layer, such as at least 0.5% by weight of the second layer, such as at least 1% by weight of the second layer, such as at least 2% by weight of the second layer.

In an embodiment of the invention, the second layer comprises the pH regulating agent in an amount of 0.2 to 10% by weight of the second layer, such as 0.5 to 8% by weight of the second layer, such as 1 to 6% by weight of the second layer, such as 2 to 4% by weight of the second layer.

According to an advantageous embodiment of the invention, the pH regulating agent comprises pH regulating agent selected from the group consisting of sodium carbonate, sodium bicarbonate, potassium carbonate, potassium bicarbonate, trometamol, amino acids, di-alkali hydrogen phosphate, tri-alkali phosphate, or any combination thereof.

In an embodiment of the invention, the pH regulating agent is selected from the group consisting of sodium carbonate, sodium bicarbonate, potassium carbonate, potassium bicarbonate, trometamol, amino acids, disodium hydrogen phosphate, dipotassium hydrogen phosphate, trisodium phosphate, tripotassium phosphate, or any combination thereof.

In an embodiment of the invention, the pH regulating agent is selected from the group consisting of sodium carbonate, sodium bicarbonate, potassium carbonate, potassium bicarbonate, trometamol, amino acids, or any combination thereof.

According to an advantageous embodiment of the invention, the pH regulating agent is selected from the group consisting of sodium carbonate, sodium bicarbonate, potassium carbonate, potassium bicarbonate, or any combination thereof.

Combinations of a carbonate and a bicarbonate may be especially advantageous. Such combination may e.g. be a sodium carbonate-sodium bicarbonate buffer system, e.g. sodium carbonate and sodium bicarbonate in a weight-ratio between 5:1 and 2.5:1, preferably in a weight-ratio between 4.1:1 and 3.5:1.

According to an advantageous embodiment of the invention, the pH regulating agent comprises or consists of sodium carbonate.

In an embodiment of the invention, the pH regulating agent is selected from the group consisting of trometamol, amino acids, disodium hydrogen phosphate, dipotassium hydrogen phosphate, trisodium phosphate, tripotassium phosphate, or any combination thereof.

In an embodiment of the invention, the pH regulating agent is selected from the group consisting of trometamol, disodium hydrogen phosphate, dipotassium hydrogen phosphate, trisodium phosphate, tripotassium phosphate, or any combination thereof.

In an embodiment of the invention, the pH regulating agent comprises di-alkali hydrogen phosphate and/or tri-alkali phosphate, such as disodium phosphate, dipotassium phosphate, trisodium phosphate and/or tripotassium phosphate.

Trometamol and phosphate buffers have a desirable relative neutral taste, hence the use of these pH regulating agents may be found not compromise the taste and mouthfeel of the nicotine tablet.

In an embodiment of the invention, the pH regulating agent comprises trometamol.

In an embodiment of the invention, the pH regulating agent consists of trometamol.

In the present context the term trometamol refers to (tris(hydroxymethyl)aminomethane), also sometimes referred to as tris buffer.

In an embodiment of the invention, the pH regulating agent comprises amino acid.

In an embodiment of the invention, the pH regulating agent consists of amino acid.

According to an advantageous embodiment of the invention, the first layer comprises sugar alcohol in an amount of at least 5% by weight of the first layer, such as at least 8% by weight of the first layer, such as at least 10% by weight of the first layer, such as at least 15% by weight of the first layer.

In an embodiment of the invention, the at least one sugar alcohol of the first layer comprises sugar alcohol in an amount of 5 to 50% by weight of the first layer, such as 8 to 40% by weight of the first layer, such as 10 to 30% by weight of the first layer, such as 15 to 30% by weight of the first layer.

According to an advantageous embodiment of the invention, the first layer comprises sugar alcohol in an amount of no more than 50% by weight of the first layer, such as no more than 40% by weight of the first layer, such as no more than 30% by weight of the first layer.

According to an advantageous embodiment of the invention, the second layer comprises sugar alcohol in an amount of at least 10% by weight of the second layer, such as at least 20% by weight of the second layer, such as at least 30% by weight of the second layer, such as at least 40% by weight of the second layer.

In an embodiment of the invention, the second layer comprises sugar alcohol in an amount of 10 to 90% by weight of the second layer, such as 20 to 80% by weight of the second layer, such as 30 to 70% by weight of the second layer, such as 40 to 60% by weight of the second layer.

According to an advantageous embodiment of the invention, the at least one sugar alcohol of the first layer comprises sugar alcohol selected from the group consisting of xylitol, maltitol, mannitol, erythritol, isomalt, sorbitol, lactitol and any combination thereof.

In an embodiment of the invention, the at least one sugar alcohol of the first layer is selected from the group consisting of xylitol, maltitol, mannitol, erythritol, isomalt, sorbitol, lactitol and any combination thereof.

In an embodiment of the invention, the at least one sugar alcohol of the first layer comprises sugar alcohol selected from the group consisting of xylitol, maltitol, erythritol, isomalt, sorbitol, lactitol and any combination thereof.

In an embodiment of the invention, the at least one sugar alcohol of the first layer is selected from the group consisting of xylitol, maltitol, erythritol, isomalt, sorbitol, lactitol and any combination thereof.

In an embodiment of the invention, the at least one sugar alcohol of the first layer comprises sorbitol and/or xylitol, such as consists of sorbitol and/or xylitol.

In an embodiment of the invention, the at least one sugar alcohol of the first layer comprises sorbitol, such as consists of sorbitol.

In an embodiment of the invention, the at least one sugar alcohol of the first layer comprises xylitol, such as consists of xylitol.

According to an advantageous embodiment of the invention, the first layer comprises water-insoluble components in an amount of no more than 30% by weight of the first layer, such as no more than 20% by weight of the first layer, such as no more than 10% by weight of the first layer.

According to an embodiment of the invention, the first layer comprises water-insoluble components in an amount of 0 to 30% by weight of the first layer, such as 0.1 to 20% by weight of the first layer, such as 1 to 10% by weight of the first layer.

As used herein the term “water-soluble” refers to a relatively high water-solubility, for example a water-solubility of more than 5 gram of water-soluble composition or substance per 100 mL of water measured at 25 degrees Celsius, atmospheric pressure and pH of 7.0. Here, atmospheric pressure refers to a pressure of about 1 atmosphere, i.e. 101,325 Pascal (Pa) or a pressure within the range of 90,000 to 110,000 Pascal (Pa).

When referring to a “soluble” composition or substance, water-soluble is meant, unless otherwise stated.

Similarly, the term “water-insoluble” refers to a relatively low water-solubility, for example a water-solubility of less than 5 gram of water-soluble composition or substance per 100 mL of water measured at 25 degrees Celsius, atmospheric pressure and pH of 7.0. Here, atmospheric pressure refers to a pressure of about 1 atmosphere, i.e. 101,325 Pascal (Pa) or a pressure within the range of 90,000 to 110,000 Pascal (Pa).

When referring to a “insoluble” composition or substance, water-insoluble is meant, unless otherwise stated.

According to an advantageous embodiment of the invention, the at least one sugar alcohol of the second layer comprises sugar alcohol selected from the group consisting of xylitol, maltitol, mannitol, erythritol, isomalt, sorbitol, lactitol and any combination thereof.

In an embodiment of the invention, the at least one sugar alcohol of the second layer is selected from the group consisting of xylitol, maltitol, mannitol, erythritol, isomalt, sorbitol, lactitol and any combination thereof.

In an embodiment of the invention, the at least one sugar alcohol of the second layer comprises sugar alcohol selected from the group consisting of xylitol, maltitol, mannitol, erythritol, isomalt, sorbitol, lactitol and any combination thereof.

In an embodiment of the invention, the at least one sugar alcohol of the second layer is selected from the group consisting of xylitol, maltitol, erythritol, isomalt, sorbitol, lactitol and any combination thereof.

In an embodiment of the invention, the at least one sugar alcohol of the second layer comprises sorbitol and/or xylitol, such as consists of sorbitol and/or xylitol.

In an embodiment of the invention, the at least one sugar alcohol of the second layer comprises sorbitol, such as consists of sorbitol.

In an embodiment of the invention, the at least one sugar alcohol of the second layer comprises xylitol, such as consists of xylitol.

According to an advantageous embodiment of the invention, the first layer constitutes at least 10% by weight of the tablet, such as at least 20% by weight of the tablet, such as at least 30% by weight of the tablet.

In an embodiment of the invention, the first layer constitutes 10 to 60% by weight of the tablet, such as 20 to 50% by weight of the tablet, such as 30 to 40% by weight of the tablet.

According to an advantageous embodiment of the invention, the second layer constitutes at least 40% by weight of the tablet, such as at least 50 by weight of the tablet, such as at least 60% by weight of the tablet.

In an embodiment of the invention, the second layer constitutes 40 to 90% by weight of the tablet, such as 50 to 80% by weight of the tablet, such as 60 to 70% by weight of the tablet.

According to an advantageous embodiment of the invention, the first layer has a weight of at least 20 mg, such as at least 40 mg, such as at least 60 mg.

In an embodiment of the invention, the first layer has a weight of 20 to 200 mg, such as 40 mg to 150 mg, such as at least 60 to 100 mg.

According to an advantageous embodiment of the invention, the second layer has a weight of at least 50 mg, such as at least 75 mg, such as at least 100 mg.

In an embodiment of the invention, the second layer has a weight of 50 to 400 mg, such as 75 to 300 mg, such as 100 to 200 mg.

According to an advantageous embodiment of the invention, the tablet is a two-layer tablet.

In an embodiment of the invention, the tablet further comprises a third layer.

According to an advantageous embodiment of the invention, the tablet is a three-layer tablet.

According to an embodiment, the tablet comprises a third layer being a mucoadhesive layer comprising a mucoadhesive.

According to an embodiment, the tablet comprises a third layer being a mucoadhesive layer comprising a natural gum mucoadhesive.

In an embodiment of the invention, the tablet comprises a third layer being an oral disintegrating tablet layer.

Thus, in the above embodiments, the tablet comprises a third layer, which is an oral disintegrating tablet layer also known as an ODT layer (ODT is an abbreviation of Oral Disintegrating Tablet).

It is noted that an oral disintegrating tablet layer may also sometimes be referred to as a fast disintegrating tablet layer, or FDT-layer.

In an embodiment of the invention, the disintegrants comprise or consists of disintegrants selected from superdisintegrants, such as crosslinked polymers, such as the group consisting of crospovidone, croscarmellose, sodium starch glycolate, and any combination thereof.

In an embodiment of the invention, the tablet comprises a third layer, said third layer comprises disintegrant in an amount of 0.5-15% by weight of the third layer, such as 1-10% by weight of the third layer.

In an embodiment of the invention, the tablet comprises a third layer.

According to an advantageous embodiment of the invention, the tablet has a weight of at least 100 mg, such as at least 150 mg, such as at least 200 mg.

According to an advantageous embodiment of the invention, the tablet has a weight of no more than 600 mg, such as no more than 500 mg, such as no more than 400 mg.

In an embodiment of the invention, the tablet has a weight of 100 to 600 mg, such as 150 to 500 mg, such as 200 to 400 mg.

According to an advantageous embodiment of the invention, the first layer has a surface, at least a part of which being substantially flat or concave.

In an embodiment of the invention, the first layer has a flat surface.

In an embodiment of the invention, the first layer has a concave surface.

In an embodiment of the invention, the second layer has a convex surface.

According to an advantageous embodiment of the invention, the first layer has a surface comprising at least one dimple.

In an embodiment of the invention, the first layer has a flat surface comprising one or more dimples.

In an embodiment of the invention, the second layer has a convex surface.

According to an advantageous embodiment of the invention, the nicotine tablet comprises flavor.

The flavor may advantageously be used as taste masking nicotine.

In an embodiment of the invention, the tablet comprises flavor in an amount of at least 0.1% by weight of the tablet.

In an embodiment of the invention, the tablet comprises flavor in an amount of 0.1 to 15.0% by weight of the tablet, such as 0.1 to 10.0% by weight of the tablet, such as 0.1 to 5.0% by weight of the tablet, such as 0.2 to 3.0% by weight of the tablet.

In an embodiment of the invention, the flavor is comprised in the first and second layer, i.e. both layers comprise flavor.

In an embodiment of the invention, the first layer comprises flavor.

In an advantageous embodiment of the invention, the flavor is comprised in the first layer.

In an embodiment of the invention, the second layer comprises flavor.

In an advantageous embodiment of the invention, the flavor is comprised in the second layer.

In an embodiment of the invention, the first layer is free of flavor.

In an embodiment of the invention, the tablet has a first ratio between content in weight percent of flavor in the first layer and the content in weight percent of nicotine in the first layer, a second ratio between content in weight percent of flavor in the second layer and the content in weight percent of nicotine in the second layer, where the first ratio is smaller than the second ratio.

In an embodiment of the invention, the tablet has a first ratio between content in weight percent of nicotine in the first layer and the content in weight percent of flavor in the first layer, a second ratio between content in weight percent of nicotine in the second layer and the content in weight percent of flavor in the second layer, where the first ratio is greater than the second ratio.

In an embodiment of the invention, the tablet comprises high intensity sweetener.

In an embodiment of the invention, the high intensity sweetener is selected from sucralose, aspartame, salts of acesulfame, such as acesulfame potassium, alitame, saccharin and its salts, cyclamic acid and its salts, glycyrrhizin, dihydrochalcones, thaumatin, monellin, stevioside, and any combination thereof.

In an embodiment of the invention, the first layer comprises high intensity sweetener.

In an embodiment of the invention, the second layer comprises high intensity sweetener.

According to an advantageous embodiment of the invention, the second layer is free of effervescent agents.

In an embodiment of the invention, the tablet is free of effervescent agents.

According to an advantageous embodiment of the invention, the first layer comprises one or more binders.

An advantage of the above embodiment may be that the dissolution rate of the first layer may be modified, i.e. the binder may be used as a dissolution modifier. Thereby, the dissolution rate of the first layer may be adjusted relative to the dissolution rate of the second layer, preferably by approaching similar dissolution rates of the first and second layers. Thus, in an advantageous embodiment of the invention, the dissolution time of the first layer is between half the dissolution time of the second layer and twice the dissolution time of the second layer.

Binder may advantageously be included in the first layer, whereby a desirable cohesiveness during tableting is achieved.

As used herein, the term “binder” refers to an ingredient promoting cohesiveness to the powder composition during tablet production and thereby facilitating production of layers and thereby tablets with a desirable mechanical strength. In an embodiment of the invention, the binders may be selected from cellulose and cellulose derivatives.

According to an advantageous embodiment of the invention, the second layer comprises less than 15% by weight of binder, such as less than 10% by weight of binder, such as less than 5% by weight of binder, such as less than 1% by weight of binder, such as less than 0.2% by weight of binder, such as being substantially free of binder.

As previously noted, low amounts of mucoadhesive may be considered as binders or dissolution modifiers, thus, in the above embodiment, the binder of the above embodiment may correspond to compounds having mucoadhesive properties if present amounts above 15% by weight of the second layer.

According to an advantageous embodiment of the invention, the second layer is free of binder.

As previously noted, low amounts of mucoadhesive may be considered as binders or dissolution modifiers, thus, in the above embodiment, the binder of the above embodiment may correspond to compounds having mucoadhesive properties if present amounts above 15% by weight of the second layer.

According to an embodiment of the invention, the first layer comprises binder in an amount of at least 1% by weight of the first layer, such as at least 2% by weight of the first layer, such as at least 4% by weight of the first layer.

According to an advantageous embodiment of the invention, the first layer comprises binder in an amount of 1 to 15% by weight of the first layer, such as 2 to 10% by weight of the first layer, such as 4 to 8% by weight of the first layer.

According to an embodiment of the invention, the first layer comprises binder in an amount of 1 to 20% by weight of the first layer, such as 2 to 15% by weight of the first layer, such as 4 to 10% by weight of the first layer.

According to an embodiment of the invention, the binder of the first layer comprises at least one binder selected from the group consisting of cellulose, carboxymethylcellulose and salts thereof, ethylcellulose, hydroxyethyl cellulose, hydroxyethyl methyl cellulose, hydroxypropyl cellulose, methylcellulose, hydroxypropyl methylcellulose, microcrystalline cellulose, poly(acrylic acid) polymers, such as carbomer and polycarbophil, polyvinylpyrrolidone (PVP), polyvinylalcohol (PVA), polyhydroxyethylmethylacrylate, polyethylene glycol, and any combination thereof.

According to an advantageous embodiment of the invention, the binder of the first layer comprises at least one binder selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

According to an advantageous embodiment of the invention, the binder of the first layer comprises at least one binder selected from the group consisting of hydroxypropyl cellulose, hydroxypropyl methylcellulose, microcrystalline cellulose, carboxymethylcellulose, and any combination thereof.

According to an advantageous embodiment of the invention, the second layer comprises binder.

An advantage of the above embodiment may be that the dissolution rate of the second layer may be modified, i.e. the binder may be used as a dissolution modifier. Thereby, the dissolution rate of the second layer may be adjusted relative to the dissolution rate of the first layer, preferably by approaching similar dissolution rates of the first and second layers. Thus, in an advantageous embodiment of the invention, the dissolution time of the first layer is between half the dissolution time of the second layer and twice the dissolution time of the second layer.

Binder may advantageously be included in the second layer, whereby a desirable cohesiveness during tableting is achieved.

According to an embodiment of the invention, the second layer comprises binder in an amount of at least 1% by weight of the second layer, such as at least 2% by weight of the second layer, such as at least 4% by weight of the second layer.

According to an advantageous embodiment of the invention, the second layer comprises binder in an amount of 1 to 15% by weight of the second layer, such as 2 to 10% by weight of the second layer, such as 4 to 8% by weight of the second layer.

According to an embodiment of the invention, the second layer comprises binder in an amount of 1 to 20% by weight of the second layer, such as 2 to 15% by weight of the first layer, such as 4 to 10% by weight of the second layer.

According to an embodiment of the invention, the binder of the second layer comprises at least one binder selected from the group consisting of cellulose, carboxymethylcellulose and salts thereof, ethylcellulose, hydroxyethyl cellulose, hydroxyethyl methyl cellulose, hydroxypropyl cellulose, methylcellulose, hydroxypropyl methylcellulose, microcrystalline cellulose, poly(acrylic acid) polymers, such as carbomer and polycarbophil, polyvinylpyrrolidone (PVP), polyvinylalcohol (PVA), polyhydroxyethylmethylacrylate, polyethylene glycol, and any combination thereof.

According to an advantageous embodiment of the invention, the binder of the second layer comprises at least one binder selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

According to an advantageous embodiment of the invention, the binder of the second layer comprises at least one binder selected from the group consisting of hydroxypropyl cellulose, hydroxypropyl methylcellulose, microcrystalline cellulose, carboxymethyl cellulose, and any combination thereof.

According to an advantageous embodiment of the invention, the tablet has a maximum dimension of at least 5 mm.

According to an advantageous embodiment of the invention, the first layer has an exposed surface of at least 0.2 cm2.

According to an advantageous embodiment of the invention, the second layer is not, during use, in contact with the oramucosal surface, which is in contact with the first layer.

For example, when the first layer is positioned to adhere to the gum (gingival mucosa), the second layer is free of contact with the gum.

In an embodiment of the invention, the first layer and the second layer have the same width.

In an embodiment of the invention, the first layer has a first width and the second has a second width, where the first width deviates no more than 5% from the second width, such as no more than 2%.

In an embodiment of the invention, the first width is identical to the second width.

In an embodiment of the invention, the first layer and the second layer have the same width.

According to an advantageous embodiment of the invention, the tablet has a hardness of at least 20N, such as at least 30N.

According to an embodiment of the invention, the tablet has a hardness of 20 to 200N, such as 30 to 150N.

According to an advantageous embodiment of the invention, the tablet has a water content below 2% by weight of the tablet, such as below 1% by weight of the tablet.

In an embodiment of the invention, the tablet has a water content of 0 to 2% by weight of the tablet, such as 0.01 to 1% by weight of the tablet.

It is noted that within the scope of the invention it is understood that neither the first layer nor the second layer comprises a coating.

According to an embodiment of the invention, a coating may be added to the tablet as claimed.

According to an embodiment of the invention, the tablet is free of coating.

The invention further relates to a dissolvable orally adhering nicotine tablet for sustained delivery of nicotine, the tablet comprising nicotine,
wherein the tablet is a multilayer tablet comprising a first layer and a second layer,
wherein the first layer is a mucoadhesive layer comprises comprising a mucoadhesive and at least one sugar alcohol and
wherein the second layer comprises at least one sugar alcohol.

The tablet disclosed above and according to the other tablet of the invention or any of its embodiments.

### DETAILED DESCRIPTION

As used herein, the term “nicotine” refers to nicotine in any form, including free base nicotine; nicotine salts; nicotine bound to a carrier, such as nicotine bound to ion exchange resins, nicotine bound to zeolites; nicotine bound to fibres or microspheres, nicotine bound to CaCO3, nicotine bound to sugar alcohol; and mixtures thereof. Bound is here to be understood as nicotine being ionically bound, adsorbed or absorbed onto the carrier, depending on the type of carrier.

When referring to nicotine amounts in milligram, the amounts are to be understood as the nicotine dose, i.e. the amounts refers to the amount of pure nicotine.

When referring to nicotine amounts in weight percent, the amounts are to be understood as the actual amount of the nicotine source in relation to the specified term, such as the first layer or the tablet. I.e. a first layer of 75 mg comprising nicotine bitartrate in an amount of 4% by weight of the first layer, refers to a first layer comprising 3 mg of nicotine bitartrate (i.e. 1 mg of pure nicotine).

Nicotine also covers nicotine not obtained from tobacco, often referred to as synthetic nicotine. Nicotine is included in the first layer. In embodiments nicotine is included in the first layer but not in the second layer.

As used herein the term “free-base nicotine” refers to non-protonated form of nicotine. Free-base nicotine may be provided as a liquid or as mixed with an amount of ion exchange resin; water-soluble compositions, such as sugar alcohols or water-soluble fibers; or water-insoluble fibers; or modified calcium carbonate. While free-base nicotine includes both free-base nicotine extracted from tobacco as well as synthetically manufactured free-base nicotine, the free-base nicotine is not provided in the form of tobacco or powdered tobacco.

As used herein, the term “nicotine salt” refers to nicotine in ionized form bound to a counterion.

As used herein, the term “NBT” refers to nicotine bitartrate and hydrates thereof.

As used herein, the term “%” and “percent” refers to percent by weight, unless otherwise is stated.

As used herein, the term “release of nicotine” refers to the nicotine being made bioavailable, i.e. available for absorption over the mucous membrane in the oral cavity. While some forms of nicotine require dissolution for being bioavailable, other forms may be readily absorbed into the body without dissolution. For example, in order for the nicotine to be bioavailable, the matrix of the tablet should be disintegrated. Some forms of nicotine require the nicotine to further be released from e.g. a carrier, e.g. nicotine from a nicotine-ion exchange resin such as nicotine polacrilex. Other nicotine forms, such nicotine salts, hereunder nicotine bitartrate, may readily dissolve upon disintegration of the matrix of the tablet. Still, some nicotine forms may not require dissolving. This applies for e.g. nicotine free base, which is released upon disintegration of the solid formulation matrix.

As used herein, the term “pH regulating agent” refers to agents, which active adjust and regulates the pH value of the solution to which they have been added or are to be added. In the present context, pH regulating agents do not include active ingredients.

When referring to amounts of an ingredient by terms such as “less than”, “no more than”, this generally refers to the particular ingredient being absent or present in a range from trace amounts to the specified maximum amount.

As used herein the term “flavor” is understood as having its ordinary meaning within the art. Flavor includes liquid and powdered flavors. Thus, flavors do of course not include sweeteners (such as sugar, sugar alcohols and high intensity sweeteners), or acids providing pure acidity/sourness, nor compounds providing pure saltiness (e.g. NaCl) or pure bitterness. The flavors can be natural or synthetic flavors.

Typically, the tablet may comprise ingredients selected from the group consisting of fillers, flavors, binders, disintegrants, hereunder super disintegrants, emulsifiers, antioxidants, pH regulating agents hereunder alkaline and acidic pH regulating agents, high intensity sweeteners, colors, glidants, lubricants, or any combination thereof.

In an embodiment of the invention, the disintegrants comprise or consists of disintegrants selected from the group consisting of crospovidone, croscarmellose, sodium starch glycolate, and any combination thereof.

In an advantageous embodiment of the invention, the tablet comprises bulk sweetener as filler ingredient.

In an advantageous embodiment of the invention, the first layer comprises bulk sweetener as filler ingredient.

In an advantageous embodiment of the invention, the second layer comprises bulk sweetener as filler ingredient.

In an advantageous embodiment of the invention, the first and second layers comprise bulk sweetener as filler ingredient.

The tablet may in addition to the at least one sugar alcohol of the first layer and the at least one sugar alcohol of the second layer, comprise different bulk sweeteners. Bulk sweeteners include sugar sweetener and/or sugarless sweetener.

Sugar sweeteners generally include, but are not limited to saccharide-containing components, such as sucrose, dextrose, maltose, saccharose, lactose, sorbose, dextrin, trehalose, D-tagatose, dried invert sugar, fructose, levulose, galactose, and the like, alone or in combination.

Sugarless sweeteners generally include but are not limited to sugar alcohols (also sometimes referred to as polyols) such as xylitol, maltitol, mannitol, erythritol, isomalt, sorbitol, lactitol, and combinations thereof.

Combinations of sugar and/or non-sugar sweeteners may be used in the tablet.

The bulk sweeteners may often support the flavor profile of the tablet.

In embodiment of the invention, bulk sweeteners may be supplemented with other usable fillers including as examples, magnesium and calcium carbonate, sodium sulphate, ground limestone, silicate compounds such as magnesium and aluminum silicate, kaolin and clay, aluminum oxide, silicon oxide, talc, titanium oxide, mono-, di- and tri-calcium phosphates, fibers, plant fibers, such as wheat fiber, oat fiber, pea fiber, and combinations thereof.

In an embodiment of the invention, the filler comprises an inorganic filler.

In an embodiment of the invention, the first layer comprises an inorganic filler.

In an embodiment of the invention, the inorganic filler is selected from the group consisting of magnesium carbonate, calcium carbonate, magnesium hydroxide, and any combination thereof.

In an embodiment of the invention, the inorganic filler comprises calcium carbonate.

In an embodiment of the invention, the inorganic filler is calcium carbonate.

In an embodiment of the invention, the inorganic filler comprises magnesium carbonate.

In an embodiment of the invention, the inorganic filler is magnesium carbonate.

In an embodiment of the invention, the inorganic filler comprises magnesium hydroxide.

In an embodiment of the invention, the inorganic filler is magnesium hydroxide.

In an embodiment of the invention, the first layer comprises inorganic filler in an amount of at least 2% by weight of the first layer, such as at least 4% by weight of the first layer.

In an embodiment of the invention, the first layer comprises inorganic filler in an amount of 2 to 20% by weight of the first layer, such as 4 to 10% by weight of the first layer.

In an embodiment of the invention, the first layer comprises natural gum mucoadhesive and inorganic filler with a weight ratio between the content of natural gum mucoadhesive and inorganic filler of 7:4 to 45, such as 9:5 to 35:2.

High intensity artificial sweetening agents can also be used in combination with the above bulk sweeteners. For example, high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (such as from about 0.02 to about 8% by weight).

In embodiments where the tablet comprises flavor, different flavors may be used.

Usable flavors including as examples almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry, kiwi, tropical punch, tutti frutti, vanilla, or any combination thereof.

According to an embodiment of the invention, flavor may be used as taste masking for the nicotine and/or taste masking of the alkaline pH regulating agent.

In an embodiment of the invention the tablet comprises glidant. Silicon dioxide may be used as a glidant. Other glidants usable for the tablet may also be used within the scope of the invention.

In an embodiment of the invention the tablet comprises lubricant. Magnesium stearate and/or sodium stearyl fumarate may be used as a lubricant. Other lubricants usable for the tablet may also be used within the scope of the invention.

### EXAMPLES

### Example 1

### Preparation of Tablets Comprising a First and a Second Layer.

The composition of second layer, i.e. the layer comprising at least one sugar alcohol, is prepared by pouring about half the sugar alcohol into a mixing bowl, followed by the other ingredients except lubricant, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The composition of first layer, i.e. the mucoadhesive layer, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The lubricated powder blends are sequentially transferred to the hopper of a tableting machine.

The second layer is then compressed at a compression force of about 1-5 kN, after which the first layer is fused by compression to the second layer at a compression force of about 8-15 kN. Punch used unless otherwise specified: 8.00 mm, circular, convex dimple, D tooling.

The tablets are manufactured on a lab scale machine, for example RIVA Piccola tablet press. The tablet machine is commissioned by adjusting the fill depth and compression force so the weight and hardness of tablets match the acceptance criteria. A pre-compression force could be included to avoid capping.

### Example 1A

Preparation of 3-Layer Tablets, Ie. Tablets Comprising a First, a Second and a Third Layer.

The composition of the third layer, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The composition of the second layer, i.e. the layer comprising at least one sugar alcohol, is prepared by pouring about half the sugar alcohol into a mixing bowl, followed by the other ingredients except lubricant, and finally the remaining sugar alcohol. The ingredients are tumbled/mixed with a mixer (Turbula or Duma) for 4-10 min at 49 rpm.

Lubricant is added and the ingredients are further mixed for 1-2 min at 49 rpm.

The composition of the first layer, i.e. the mucoadhesive layer, is prepared by pouring all the ingredients except lubricant, into a mixing bowl. The ingredients are tumbled/mixed with a mixer (Turbula or Duma) for 4-10 min at 49 rpm.

Lubricant is added and the ingredients are further mixed for 1-2 min at 49 rpm.

The lubricated powder blends are sequentially transferred to the hoppers of a tableting machine.

The first layer is compressed at a compression force of about 1-6 kN, after which the second layer is fused by compression to the first layer at a compression force of about 2-8 kN. Then, the third layer is fused by compression to the second layer at a compression force of about 20-40 kN. Punch used unless otherwise specified: 12.00 mm, circular, shallow concave, B tooling.

The tablets are manufactured on a PZ-TRE rotary tablet press available from B&D Italia, but other standard equipment for making tri-layer tablets may be used as well, such as a PTK PR3500 or a Hata tri-layer tablet press. The tablet machine is commissioned by adjusting the fill depth and compression force so the weight and hardness of tablets match the acceptance criteria. A pre-compression force could be included to avoid capping.

### Example 2: Tablet Compositions

### Example 2A: Nicotine Sources

150 mg tablets were made each with 85 mg second layer and 65 mg first layer. The tablets were prepared according to example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 1
Compositions of first and second layers.

| | NT1 | NT2 | NT3 | NT4 | NT5 | NT6 | NT7 | NT8 | NT9 |
|---|---|---|---|---|---|---|---|---|---|
| Nicotine dose | 1.0 | 3.0 | 3.5 | 5.2 | 1.2 | 1.0 | 2.6 | 1.5 | 1.0 |
| 1^{st} layer [mg] | | | | | | | | | |
| Raw material | Content in weight percent of 1st layer | | | | | | | | |
| 1^{st} layer | | | | | | | | | |
| Gum arabic | 60 | 60 | 60 | 60 | 60 | 60 | 60 | 60 | 60 |
| Mannitol | 29.3 | 14.7 | 7.5 | 11 | 23.3 | 24.2 | 20 | 22.5 | 29.3 |
| MgSt | 1 | 1 | 1 | 1 | 1 | 1 | 1 | 1 | 1 |
| NBT | 4.7 | 14.3 | 16.5 | — | — | — | — | — | 4.7 |
| Nicotine | — | — | — | — | 5.7 | — | — | — | — |
| MCC | | | | | | | | | |
| NPR (15%) | — | — | — | — | — | 9.8 | — | 5 | — |
| Nicotine | — | — | — | 8 | — | — | 4 | 1.5 | — |
| free base | | | | | | | | | |
| Na2CO3 | 5 | 10 | 15 | 20 | 10 | 5 | 15 | 10 | 5 |
| Total 1st layer | 100 | 100 | 100 | 100 | 100 | 100 | 100 | 100 | 100 |
| Nicotine dose | — | — | — | — | — | — | — | — | 1.0 |
| 2^{nd} layer [mg] | | | | | | | | | |
| Raw material | Content in weight percent 2^{nd} layer | | | | | | | | |
| 2^{nd} layer | | | | | | | | | |
| Xanthan gum | 2 | 2 | 2 | 2 | 2 | 2 | 2 | 2 | 2 |
| Mannitol | 92.9 | 92.9 | 92.9 | 92.9 | 92.9 | 92.9 | 92.9 | 92.9 | 84.4 |
| HIS | 0.6 | 0.6 | 0.6 | 0.6 | 0.6 | 0.6 | 0.6 | 0.6 | 0.6 |
| MgSt | 2 | 2 | 2 | 2 | 2 | 2 | 2 | 2 | 2 |
| Flavor | 2.5 | 2.5 | 2.5 | 2.5 | 2.5 | 2.5 | 2.5 | 2.5 | 2.5 |
| NBT | — | — | — | — | — | — | — | — | 3.5 |
| Na2CO3 | — | — | — | — | — | — | — | — | 5 |
| Total2^{nd} layer | 100 | 100 | 100 | 100 | 100 | 100 | 100 | 100 | 100 |

HIS = high intensity sweetener.
NBT = nicotine bi-tartrate (nicotine content of 32.38% by weight).
NPR = nicotine bound to polacrilex resin.
Nicotine MCC = free nicotine base absorbed onto a microcrystalline cellulose carrier in a weight ratio of 1:2. Thus, the nicotine makes up a third of the weight in nicotine MCC.

For samples NT4, NT7 and NT8, the nicotine was applied by means of a premix of nicotine free base and mannitol.

Preferred high intensity sweeteners (HIS) may e.g. be sucralose, acesulfame potassium, and mixtures thereof. Other high intensity sweeteners, such as aspartame, salts of acesulfame, such as acesulfame potassium, alitame, saccharin and its salts, cyclamic acid and its salts, glycyrrhizin, dihydrochalcones, thaumatin, monellin, stevioside, alone or in combination, are also usable within the scope of the invention.

Fruit flavors, and mixtures thereof, menthol, peppermint, and mixtures thereof, may be used in the above formulations as flavors. Other flavors may also be used within the scope of the invention.

In the above MgSt (magnesium stearate) is used as lubricant. Other lubricants, such as sodium stearyl fumerate may also be usable within the scope of the invention.

Sodium carbonate is used as the alkaline pH regulating agent. Further usable alkaline pH regulating agents include sodium bicarbonate, potassium carbonate, potassium bicarbonate, trometamol, amino acids, disodium hydrogen phosphate, dipotassium hydrogen phosphate, trisodium phosphate, tripotassium phosphate, or any combination thereof.

### Examples 2B: Buffer Variations

200 mg tablets were made each with 100 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 2
Compositions of first and second layers.

| | NT | NT | NT | NT | NT | NT | NT | NT |
|---|---|---|---|---|---|---|---|---|
| | 11 | 12 | 13 | 14 | 15 | 16 | 17 | 18 |
| Nicotine dose | 3.0 | 3.0 | 3.0 | 3.0 | 3.0 | 3.0 | 2.0 | 2.0 |
| 1^{st} layer [mg] | | | | | | | | |
| Raw material | Content in weight percent of 1^{st} layer | | | | | | | |
| 1^{st} layer | | | | | | | | |
| Gum arabic | 60 | 60 | 60 | 60 | 60 | 60 | 60 | 60 |
| Mannitol | 28.6 | 18.6 | 18.6 | 18.6 | 18.6 | 18.6 | 31.7 | 31.7 |
| MgSt | 1 | 1 | 1 | 1 | 1 | 1 | 1 | 1 |
| Flavor | 1 | 1 | 1 | 1 | 1 | 1 | 1 | 1 |
| NBT | 9.4 | 9.4 | 9.4 | 9.4 | 9.4 | 9.4 | 6.3 | 6.3 |
| Na2CO3 | — | — | — | — | — | 5 | — | — |
| NaHCO3 | — | 10 | — | — | — | 5 | — | — |
| Disodium | — | — | 10 | — | — | — | — | — |
| hydrogen | | | | | | | | |
| phosphate | | | | | | | | |
| K2CO3 | — | — | — | 10 | — | — | — | — |
| Trometamol | — | — | — | — | 10 | — | — | — |
| Total 1st | 100 | 100 | 100 | 100 | 100 | 100 | 100 | 100 |
| layer | | | | | | | | |
| Raw material | Content in weight percent of 2^{nd} layer | | | | | | | |
| 2^{nd} layer | | | | | | | | |
| Xanthan gum | 2 | 2 | 2 | 2 | 2 | 2 | 3 | 5 |
| Mannitol | 94.9 | 94.9 | 94.9 | 94.9 | 94.9 | 94.9 | 93.9 | 91.9 |
| HIS | 0.6 | 0.6 | 0.6 | 0.6 | 0.6 | 0.6 | 0.6 | 0.6 |
| Flavor | 2.5 | 2.5 | 2.5 | 2.5 | 2.5 | 2.5 | 2.5 | 2.5 |
| Total 2nd | 100 | 100 | 100 | 100 | 100 | 100 | 100 | 100 |
| layer | | | | | | | | |

NBT = nicotine bi-tartrate (nicotine content of 32.38% by weight).
HIS = high intensity sweetener.

Alternative ingredients as described in relation to NT1-NT9 of table 1 may also be applied for NT11-NT18.

### Example 2C: Binder Variations

150 mg tablets were made each with 85 mg second layer and 65 mg first layer. The tablets were prepared according to example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 3
Compositions of first and second layers.

| | NT | NT | NT | NT | NT | NT | NT | NT |
|---|---|---|---|---|---|---|---|---|
| | 21 | 22 | 23 | 24 | 25 | 26 | 27 | 28 |
| Nicotine dose | 2.0 | 2.0 | 2.0 | 2.0 | 2.0 | 2.0 | 2.0 | 2.0 |
| 1^{st} layer [mg] | | | | | | | | |
| Raw material | Content in weight percent of 1^{st} layer | | | | | | | |
| 1^{st} layer | | | | | | | | |
| Gum arabic | 60 | 60 | 60 | 60 | 60 | 60 | 60 | 60 |
| Mannitol | 14.6 | 9.6 | 12.1 | 12.1 | 4.6 | 19.6 | 19.6 | 19.6 |
| MgSt | 1 | 1 | 1 | 1 | 1 | 1 | 1 | 1 |
| HPC | 5 | 10 | — | — | 7.5 | — | — | — |
| MCC | — | — | 7.5 | — | 7.5 | — | — | — |
| CMC | — | — | — | 7.5 | — | — | — | — |
| NBT | 9.4 | 9.4 | 9.4 | 9.4 | 9.4 | 9.4 | 9.4 | 9.4 |
| Na2CO3 | 10 | 10 | 10 | 10 | 10 | 10 | 10 | 10 |
| Total 1st | 100 | 100 | 100 | 100 | 100 | 100 | 100 | 100 |
| layer | | | | | | | | |
| Raw material | Content in weight percent of 2^{nd} layer | | | | | | | |
| 2^{nd} layer | | | | | | | | |
| Xanthan gum | 2 | 2 | 2 | 2 | 2 | 2 | 2 | 2 |
| MgSt | 2 | 2 | 2 | 2 | 2 | 2 | 2 | 2 |
| Mannitol | 92.9 | 92.9 | 92.9 | 92.9 | 92.9 | 87.9 | 82.9 | 77.9 |
| HPC | — | — | — | — | — | 5 | — | 10 |
| MCC | — | — | — | — | — | — | 5 | — |
| CMC | — | — | — | — | — | — | 5 | 5 |
| HIS | 0.6 | 0.6 | 0.6 | 0.6 | 0.6 | 0.6 | 0.6 | 0.6 |
| Flavor | 2.5 | 2.5 | 2.5 | 2.5 | 2.5 | 2.5 | 2.5 | 2.5 |
| Total 2nd | 100 | 100 | 100 | 100 | 100 | 100 | 100 | 100 |
| layer | | | | | | | | |

HPC = hydroxypropyl cellulose.
HIS = high intensity sweetener.
NBT = nicotine bi-tartrate (nicotine content of 32.38% by weight).
MCC = microcrystalline cellulose.
CMC = carboxymethyl cellulose.

Alternative ingredients as described in relation to NT1-NT18 of table 1 may also be applied for NT21-NT28.

### Example 2D Polyol Variations

300 mg tablets were made each with 200 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 4
Compositions of first and second layers.

| | NT31 | NT32 | NT33 | NT34 | NT35 | NT36 | C2 |
|---|---|---|---|---|---|---|---|
| Nicotine dose | 3.0 | 3.0 | 3.0 | 3.0 | 3.0 | 3.0 | 3.0 |
| 1^{st} layer [mg] | | | | | | | |
| Raw material | Content in weight percent of 1^{st} layer | | | | | | |
| 1^{st} layer | | | | | | | |
| Gum arabic | 60 | 60 | 60 | 60 | 60 | 60 | 10 |
| HPC | — | — | — | — | — | 10 | 10 |
| Xylitol | 19.6 | — | — | — | — | — | — |
| Isomalt | — | 19.6 | — | — | — | — | — |
| Maltitol | — | — | 19.6 | — | — | — | — |
| Erythtritol | — | — | — | 10 | — | — | — |
| Sorbitol | — | — | — | 9.6 | 19.6 | 9.6 | 59.6 |
| MgSt | 1 | 1 | 1 | 1 | 1 | 1 | 1 |
| NBT | 9.4 | 9.4 | 9.4 | 9.4 | 9.4 | 9.4 | 9.4 |
| Na2CO3 | 10 | 10 | 10 | 10 | 10 | 10 | 10 |
| Total 1^{st} layer | 100 | 100 | 100 | 100 | 100 | 100 | 100 |
| Raw material | Content in weight percent of 2^{nd} layer | | | | | | |
| 2^{nd} layer | | | | | | | |
| Mannitol | 96.5 | — | — | — | 46.5 | — | — |
| Isomalt | — | 96.5 | — | — | — | — | — |
| Maltitol | — | — | 96.5 | — | — | — | — |
| Erythtritol | — | — | — | 96.5 | — | — | — |
| Sorbitol | — | — | — | — | 50 | 88 | 88 |
| HPC | — | — | — | — | — | 10 | 10 |
| MgSt | 1 | 1 | 1 | 1 | 1 | 0.5 | 0.5 |
| Flavor | 2.5 | 2.5 | 2.5 | 2.5 | 2.5 | 1.5 | 1.5 |
| Total 2^{nd} layer | 100 | 100 | 100 | 100 | 100 | 100 | 100 |

NBT = nicotine bi-tartrate (nicotine content of 32.38% by weight).
HPC = hydroxypropyl cellulose.

Alternative ingredients as described in relation to NT1-NT28 of table 1 may also be applied for NT31-NT36.

### Example 2E: Mucoadhesive Variations

150 mg tablets were made each with 75 mg second layer and 75 mg first layer. The tablets were prepared according to example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 5
Compositions of first and second layers.

| | NT41 | NT42 | NT43 | NT44 | NT45 | NT46 | NT47 |
|---|---|---|---|---|---|---|---|
| Nicotine dose | 1.0 | 1.0 | 1.0 | 3.0 | 3.0 | 3.0 | 3.0 |
| 1^{st} layer [mg] | | | | | | | |
| Raw material | Content in weight percent of 1^{st} layer | | | | | | |
| 1^{st} layer | | | | | | | |
| Gum arabic | 50 | 70 | 90 | — | 60 | 60 | 20 |
| Xanthan gum | — | — | — | 60 | — | — | — |
| Mannitol | 35 | 15 | 3 | 16.5 | 16.3 | 14.3 | 56.5 |
| MgSt | 1 | 1 | 1 | 1 | 1 | 1 | 1 |
| Flavor | — | — | — | — | — | 2 | — |
| HIS | — | — | — | — | 0.2 | 0.2 | — |
| NBT | 4 | 4 | 4 | 12.5 | 12.5 | 12.5 | 12.5 |
| Na2CO3 | 10 | 10 | 2 | 10 | 10 | 10 | 10 |
| Total 1^{st} layer | 100 | 100 | 100 | 100 | 100 | 100 | 100 |
| Raw material | Content in weight percent of 2^{nd} layer | | | | | | |
| 2^{nd} layer | | | | | | | |
| Gum arabic | 2 | 2 | 2 | — | — | — | — |
| Xanthan gum | — | — | — | 2 | 2 | 2 | 2 |
| Mannitol | 93.9 | 93.9 | 93.9 | 93.9 | 93.9 | 93.9 | 93.9 |
| HIS | 0.6 | 0.6 | 0.6 | 0.6 | 0.6 | 0.6 | 0.6 |
| MgSt | 2 | 2 | 2 | 2 | 2 | 2 | 2 |
| Flavor | 1.5 | 1.5 | 1.5 | 1.5 | 1.5 | 1.5 | 1.5 |
| Total 2^{nd} layer | 100 | 100 | 100 | 100 | 100 | 100 | 100 |

HPC = hydroxypropyl cellulose.
HIS = high intensity sweetener.
NBT = nicotine bi-tartrate (nicotine content of 32.38% by weight).

Alternative ingredients as described in relation to NT1-NT35 may also be applied for NT41-NT47.

### Example 2F: Mucoadhesive Variations

150 mg tablets were made each with 85 mg second layer and 65 mg first layer. The tablets were prepared according to example 1.

Punch used: 8.00 mm, circular, convex dimple, D tooling.

The second layer is compressed at a compression force of about 1-5 kN, after which the first layer is fused by compression to the second layer at a compression force of about 8-15 kN.

TABLE 6
Compositions of first and second layers.

| | NT51 | NT52 | NT53 | NT54 | NT55 |
|---|---|---|---|---|---|
| Nicotine dose 1^{st} layer [mg] | 3.0 | 3.0 | 3.0 | 3.0 | 3.0 |
| Raw material 1^{st} layer | Content in weight percent of 1^{st} layer | | | | |
| Gum arabic | 60 | — | 30 | 40 | 30 |
| HPMC | — | 60 | 30 | 20 | — |
| Pearlitol CR H-EXP | — | — | — | — | 40 |
| Mannitol | 14.7 | 14.7 | 14.7 | 14.7 | 4.7 |
| MgSt | 1 | 1 | 1 | 1 | 1 |
| NBT | 14.3 | 14.3 | 14.3 | 14.3 | 14.3 |
| Na2CO3 | 10 | 10 | 10 | 10 | 10 |
| Total 1^{st} layer | 100 | 100 | 100 | 100 | 100 |
| Raw material 2^{nd} layer | Content in weight percent of 2^{nd} layer | | | | |
| Xanthan | 2 | 2 | 2 | 2 | 2 |
| Mannitol | 92.9 | 92.9 | 92.9 | 92.9 | 92.9 |
| HIS | 0.6 | 0.6 | 0.6 | 0.6 | 0.6 |
| MgSt | 2 | 2 | 2 | 2 | 2 |
| Flavor | 2.5 | 2.5 | 2.5 | 2.5 | 2.5 |
| Total 2^{nd} layer | 100 | 100 | 100 | 100 | 100 |

HPMC = Hydropropyl methylcellulose.
HIS = high intensity sweetener.
NBT = nicotine bi-tartrate (nicotine content of 32.38% by weight).
Pearlitol CR H-EXP is a co-processed mannitol hydroxypropyl methylcellulose available from Roquette.

Alternative ingredients as described in relation to NT1-NT47 may also be applied for NT51-NT55.

### Example 2G

Tablets with varying size were prepared according to example 1A.

Punch used for samples NT61-NT66:12.00 mm, circular, shallow concave, B tooling.

The first layer is compressed at a compression force of about 1-6 kN, after which the second layer is fused by compression to the first layer at a compression force of about 2-8 kN. Then, the third layer is fused by compression to the second layer at a compression force of about 20-40 kN.

TABLE 6A
Compositions of first and second layers.

| | NT61 | NT62 | NT63 | NT64 | NT65 | NT66 |
|---|---|---|---|---|---|---|
| 1st layer weight [mg] | 100 | 100 | 100 | 100 | 100 | 100 |
| Nicotine dose 1st | 2 | 2 | 2 | 2 | 2 | 2 |
| layer [mg] | | | | | | |
| Raw material 1^{st} layer | Content in weight percent of 1st module | | | | | |
| CaCO3 | 10 | 10 | 10 | 10 | 10 | 10 |
| HPC | 5 | 5 | 5 | 5 | 5 | 5 |
| NBT | 6.3 | 6.3 | 6.3 | 6.3 | 6.3 | 6.3 |
| Gum arabic | 77.7 | 77.7 | 77.7 | 77.7 | 77.7 | 77.7 |
| MgSt | 1 | 1 | 1 | 1 | 1 | 1 |
| Total 1^{st} layer | 100 | 100 | 100 | 100 | 100 | 100 |
| 2nd layer weight [mg] | 300 | 300 | 300 | 300 | 300 | 300 |
| Nicotine dose 2^{nd} | 1 | 1 | 1 | 1 | 1 | 1 |
| layer [mg] | | | | | | |
| Raw material 2^{nd} layer | Content in weight percent of 2^{nd} module | | | | | |
| Xylitol | 85.8 | 85.8 | 77.8 | 77.8 | 81.8 | 81.8 |
| Xanthan gum | — | — | 6 | 6 | 3 | 3 |
| Sodium alginate | — | — | 2 | 2 | 1 | 1 |
| NBT | 1 | 1 | 1 | 1 | 1 | 1 |
| Na2CO3 | 1.5 | 1.5 | 1.5 | 1.5 | 1.5 | 1.5 |
| HIS | 0.2 | 0.2 | 0.2 | 0.2 | 0.2 | 0.2 |
| HPC | 10 | 10 | 10 | 10 | 10 | 10 |
| Flavor | 1.5 | 1.5 | 1.5 | 1.5 | 1.5 | 1.5 |
| Total 2^{nd} layer | 100 | 100 | 100 | 100 | 100 | 100 |
| 3^{rd} layer weight [mg] | 100 | 100 | 100 | 100 | 100 | 100 |
| Nicotine dose 3^{rd} | 1 | 1 | 1 | 1 | 1 | 1 |
| layer [mg] | | | | | | |
| Raw material 3^{rd} layer | Content in weight percent of 3^{rd} module | | | | | |
| Pearlitol flash | 94.3 | 54.3 | 94.3 | 54.3 | 56.3 | 94.3 |
| Xylitol | — | 40 | — | 40 | 30 | — |
| Xanthan gum | — | — | — | — | 6 | — |
| Sodium alginate | — | — | — | — | 2 | — |
| NBT | 3.1 | 3.1 | 3.1 | 3.1 | 3.1 | 3.1 |
| HIS | 0.1 | 0.1 | 0.1 | 0.1 | 0.1 | 0.1 |
| HPC | 1 | 1 | 1 | 1 | 1 | 1 |
| Flavor | 0.5 | 0.5 | 0.5 | 0.5 | 0.5 | 0.5 |
| Total 3^{rd} layer | 100 | 100 | 100 | 100 | 100 | 100 |

HPC = hydroxypropyl cellulose.
HIS = high intensity sweetener.
NBT = nicotine bi-tartrate (nicotine content of 32.38% by weight.

In table 6A, sucralose was used as high intensity sweetener. Flavor may e.g. be combination of peppermint and menthol.

It is noted that Pearlitol flash is a commercially available ready to use system comprising mannitol and starch in a mannitol to starch weight ratio of approximately 4:1.

Alternative ingredients as described in relation to NT1-NT55 may also be applied for NT61-NT66.

### Example 2H

400 mg tablets were made each with 300 mg second layer and 100 mg first layer. The tablets were prepared according to example 1.

Punch used: 12.00 mm, circular, shallow concave, B tooling.

The second layer is compressed at a compression force of about 3-8 kN, after which the first layer is fused by compression to the second layer at a compression force of about 20-30 kN.

TABLE 6B
Compositions of first and second layers.

| | NT71 | NT72 | NT73 | NT74 | NT75 | NT76 |
|---|---|---|---|---|---|---|
| Nicotine dose 1^{st} layer [mg] | 2 | 2 | 2 | 2 | 2 | 2 |
| Raw material 1^{st} layer | Content in weight percent of 1st module | | | | | |
| Sorbitol | 17.7 | 17.7 | 17.7 | 39.7 | 42.7 | 5.7 |
| Calcium carbonate | 6 | 6 | 6 | 4 | 6 | 8 |
| HPC | 5 | 5 | 5 | 5 | 5 | 5 |
| NBT | 6.3 | 6.3 | 6.3 | 6.3 | 6.3 | 6.3 |
| Na2CO3 | 4 | 4 | 4 | 4 | 4 | 4 |
| Gum arabic | 60 | 60 | 60 | 40 | 35 | 70 |
| MgSt | 1 | 1 | 1 | 1 | 1 | 1 |
| Total 1^{st} layer | 100 | 100 | 100 | 100 | 100 | 100 |
| Raw material 2^{nd} layer | Content in weight percent of 2^{nd} module | | | | | |
| Xylitol | 98.3 | 93.3 | 88.3 | 88.3 | 88.3 | 88.3 |
| HIS | 0.2 | 0.2 | 0.2 | 0.2 | 0.2 | 0.2 |
| HPC | — | 5 | 10 | 10 | 10 | 10 |
| Flavor | 0.5 | 0.5 | 0.5 | 0.5 | 0.5 | 0.5 |
| MgSt | 1 | 1 | 1 | 1 | 1 | 1 |
| Total 2^{nd} layer | 100.0 | 100.0 | 100.0 | 100.0 | 100.0 | 100.0 |

HPC = hydroxypropyl cellulose.
HIS = high intensity sweetener.
NBT = nicotine bi-tartrate (nicotine content of 32.38% by weight.

In table 6B, sucralose was used as high intensity sweetener. Flavor may e.g. be combination of peppermint and menthol.

Alternative ingredients as described in relation to NT1-NT66 may also be applied for NT71-NT76.

### Example 3: Evaluation of Tablets

For evaluation a panel of 7 trained assessors was used. Each assessor repeated the evaluations twice.

The trained assessors abstain from eating and drinking at least 30 minutes before initiation of any test. Each trained assessor was a healthy person appointed on an objective basis according to specified requirements.

For testing the tablet was weighted and placed in the mouth, between the upper lip and the gum, with the first layer, i.e. the mucoadhesive layer, facing the gum. At specific time points, e.g. 15, 30 and 60 minutes, the content of nicotine was measured in the remaining tablet residue, if still present. Once the desired test time was achieved, the tablet was taken out and weighed directly into a measuring glass to be used for analysis of nicotine content. The nicotine content was analyzed by means of standard HPLC technique after extraction into relevant buffer.

Optionally, the tablets were evaluated with respect to mouth feel, taste and other sensory parameters during testing.

If dissolution of the tablet is substantially complete before the specified time point was reached, the time of dissolution was registered as the in vivo dissolution time of the tablet.

The evaluated tablets were found to be highly suitable delivery vehicles for nicotine, giving desirable mouthfeel and taste profile while providing a surprisingly sustained release of nicotine.

In particular, the evaluation panel reported a surprisingly effective release of nicotine from tested NT2 tablets, yet only very low levels of nicotine burning was detected by the evaluation panel to the surprise of the inventors.

### Example 4: In Vivo Dissolution Time

Dissolution time of NT2 tablet was tested according to example 3.

The dissolution time of selected tablets were tested and is shown in below table 7.

TABLE 7
In vivo dissolution time

| Tablet | NT2 |
|---|---|
| In vivo dissolution time [min] | 30 |

### Example 5. Adhering of Tablets

Tablet NT36 and comparative tablet C2 of example 2D were evaluated with respect to adhesiveness.

A panel of 6 trained assessors was used. The trained assessors abstain from eating and drinking at least 30 minutes before initiation of any test. Each trained assessor was a healthy person appointed on an objective basis according to specified requirements.

Assessors chosen in accordance with example 3 were instructed to insert tablet between the gum and lip with the adhesive first layer facing the gum.

The adhesiveness of the tablets was evaluated after 1 minute and again after 5 minutes after insertion. Every assessor gave the same answer, which is shown in table 8 below.

TABLE 8
Evaluation of adhesiveness: did the tablet
adhere to the gum at the specified time.

| Time [min] | 1 | 5 |
|---|---|---|
| Tablet | Adhesion evaluation | |
| NT36 | Yes | Yes |
| C2 | No | No |

As can be seen from table 8, comparative table C2 is not adhesive due to the very low content of mucoadhesive (only 10%) in the first layer. Inventive tablet NT36, with a sufficient content of natural gum mucoadhesive in the first layer is adhesive both after 1 minute and after 5 minutes after insertion on the gum.

### Example 6. Evaluation

Tablets NT61-NT66 were evaluated with respect to dissolution time and mouthfeel.

The in vivo dissolution time (as determined according to Example 3) of tablets NT61-NT66, which all have the same composition of the adhesive first layer and the same level of binder in non-adhesive second layer, was determined to be in the range of 130-150 min for all samples.

However, it was noticed that approximately the same overall in vivo dissolution time was observed despite significant differences in dissolution of the fast dissolving third layer comprising Pealitol Flash. In vivo dissolution of the third layer ranged from 5 to 8 minutes for samples NT61, NT63 and NT66 with the highest contents of Pearlitol Flash, whereas an in vivo dissolution time of the third layer of 10 to 15 minutes was observed for samples NT62, NT64 and NT65 containing a blend of Pearlitol Flash and xylitol.

### Example 7. In Vivo Dissolution Time

The in vivo dissolution time (as determined according to Example 3) of selected tablets was tested and is shown in below table 9.

TABLE 9
In vivo dissolution time

| Tablet | NT71 | NT72 | NT73 |
|---|---|---|---|
| In vivo dissolution time [min] | 107 | 122 | 151 |

As seen from table 9, the dissolution time of the tablets could be gradually varied by adjusting the amount of binder, e.g. between 0 and 10% in NT71-NT73.

1. A dissolvable orally adhering nicotine tablet for sustained delivery of nicotine, wherein the tablet is a multilayer tablet comprising a first layer and a second layer,
wherein the first layer is a mucoadhesive layer comprising a mucoadhesive, nicotine, and at least one sugar alcohol, and
wherein the second layer comprises at least one sugar alcohol.

2. The tablet according to claim 1, wherein the tablet is a compressed tablet.

3-4. (canceled)

5. The tablet according to claim 1, wherein the mucoadhesive comprises a mucoadhesive selected from the group consisting of xanthan gum, konjac gum, tara gum, gellan gum, locust bean gum, gum arabic, alginic acid, alginate, pullulan, tragacanth gum, gum karaya, fenugreek gum, cassia gum, carrageenan, agar, pectin, hydroxypropyl cellulose (HPC), hydroxypropyl methylcellulose (HPMC), hydroxy ethylcellulose, ethylcellulose (EC), carboxymethyl cellulose (CMC) and salts thereof, dextran, guar gum, polyvinyl pyrrolidone (PVP), gelatin, casein, acrylic acid polymers (carbomers), acrylic acid esters, acrylic acid copolymers, and any combination thereof.

6-13. (canceled)

14. The tablet according to claim 1, wherein the first layer comprises at least two different types of mucoadhesives.

15-16. (canceled)

17. The tablet according to claim 1, wherein the first layer comprises the mucoadhesive in an amount of at least 35% by weight of the first layer.

18-20. (canceled)

21. The tablet according to claim 1, wherein the tablet comprises nicotine in an amount of at least 0.2% by weight of the tablet.

22-23. (canceled)

24. The tablet according to claim 1, wherein the nicotine is selected from the group consisting of a nicotine salt, nicotine free base, a nicotine-ion exchange resin combination, a nicotine inclusion complex or nicotine in any non-covalent binding; nicotine bound to zeolites, nicotine bound to cellulose, starch microspheres, and mixtures thereof.

25-30. (canceled)

31. The tablet according to claim 1, wherein the tablet has a dissolution time of at least 15 minutes.

32. (canceled)

33. The tablet according to claim 1, wherein the tablet comprises a pH regulating agent.

34. The tablet according to claim 1, wherein the tablet comprises the pH regulating agent in an amount of at least 0.2% by weight of the tablet.

35. The tablet according to claim 1, wherein the pH regulating agent is comprised in the first layer.

36. (canceled)

37. The tablet according to claim 1, wherein the pH regulating agent comprises pH regulating agent selected from the group consisting of sodium carbonate, sodium bicarbonate, potassium carbonate, potassium bicarbonate, trometamol, amino acids, di-alkali hydrogen phosphate, tri-alkali phosphate, or any combination thereof.

38-39. (canceled)

40. The tablet according to claim 1, wherein the first layer comprises sugar alcohol in an amount of at least 5% by weight of the first layer.

41. (canceled)

42. The tablet according to claim 1, wherein the second layer comprises sugar alcohol in an amount of at least 10% by weight of the second layer.

43. The tablet according to claim 1, wherein the at least one sugar alcohol of the first layer comprises sugar alcohol selected from the group consisting of xylitol, maltitol, mannitol, erythritol, isomalt, sorbitol, lactitol and any combination thereof.

44. The tablet according to claim 1, wherein the at least one sugar alcohol of the second layer comprises sugar alcohol selected from the group consisting of xylitol, maltitol, mannitol, erythritol, isomalt, sorbitol, lactitol and any combination thereof.

45-53. (canceled)

54. The tablet according to claim 1, wherein the first layer has a surface comprising at least one dimple.

55-60. (canceled)

61. The tablet according to claim 1, wherein the second layer comprises binder.

62. The tablet according to claim 1, wherein the second layer comprises less than 15% by weight of binder.

63-64. (canceled)

65. The tablet according to claim 1, wherein the binder of the second layer comprises at least one binder selected from the group consisting of cellulose, carboxymethylcellulose, ethylcellulose, hydroxyethyl cellulose, hydroxyethyl methyl cellulose, hydroxypropyl cellulose, methylcellulose, hydroxypropyl methylcellulose, microcrystalline cellulose, and any combination thereof.

66-73. (canceled)

74. A dissolvable orally adhering nicotine tablet for sustained delivery of nicotine, wherein the tablet is a multilayer tablet comprising a first layer and a second layer,
wherein the first layer is a mucoadhesive layer comprising a mucoadhesive, nicotine, and at least one sugar alcohol
wherein the second layer comprises at least one sugar alcohol, and
wherein the second layer comprises nicotine.

75. A dissolvable orally adhering nicotine tablet for sustained delivery of nicotine, wherein the tablet is a multilayer tablet comprising a first layer and a second layer,
wherein the first layer is a mucoadhesive layer comprising a mucoadhesive, nicotine, and at least one sugar alcohol
wherein the second layer comprises at least one sugar alcohol, and
wherein the mucoadhesive comprises gum arabic.
